(19) United States
(12) Patent Application Publication
(10) Pub. No.: US 2020/0108227 A1
(43) Pub. Date: Apr. 9, 2020

(54) PROPULSION AND CONTROL OF A MICRO-DEVICE

(71) Applicants: Michael SHPIGELMACHER, Los Angeles, CA (US); BIONAUT LABS LTD., Herzliya (IL)

(72) Inventors: Michael SHPIGELMACHER, Los Angeles, CA (US); Alex KISELYOV, San Diego, CA (US)

(73) Assignee: BIONAUT LABS LTD., Herzliya (IL)

(21) Appl. No.: 16/609,493

(22) PCT Filed: May 3, 2018
(86) PCT No.: PCT/US2018/030942

§ 371 Date: Oct. 30, 2019

Related U.S. Application Data
(60) Provisional application No. 62/501,156, filed on May 4, 2017.

(51) Int. Cl. A61M 25/01; A61B 5/00; A61N 1/05; A61N 2/00; A61N 5/10; A61N 7/00; A61M 25/00

(52) U.S. Cl. CPC A61M 25/0116; A61B 5/6861; A61N 1/05; A61N 2/002; A61M 31/002; A61N 7/00; A61M 25/0127; A61M 25/0122; A61M 25/0074; A61N 5/1002

(57) ABSTRACT

A device configured to move in a viscoelastic media, the device comprising: a main-body comprising a first material, configured to respond to a first threshold of a stimulus field; and one or more memory shaped elements comprising a second material, configured to respond to a second threshold of a stimuli field; wherein the first material is selected to enable manipulation of the main-body's direction in the viscoelastic media; and wherein second material is selected to enable manipulation of the configuration of the memory shaped element.

### CROSS REFERENCE TO RELATED APPLICATIONS

This application claims benefit of U.S. Provisional Patent Application Ser. No. 62/501,156, filed May 4, 2017, the priority date of which is hereby claimed.

### BACKGROUND OF THE INVENTION

A number of techniques have been proposed for magnetically-actuated propulsion of microscopic objects (sometimes referred to as micro-robots). For example, U.S. Pat. No. 8,768,501, which is incorporated herein by reference, describes methods and systems for the fabrication and application of magnetically actuated propellers (MAPs), with typical feature size in the range of 20 nanometers up to 100 microns (micrometers), in one spatial dimension.

Another technique for magnetic actuation is the selective and directional actuation of elastomer films, utilizing magnetic anisotropy introduced by chains of Fe₃O₄ magnetic nanoparticles (MNPs). See Mishra et al., “Selective and directional actuation of elastomer films using chained magnetic nanoparticles,” Nanoscale 8 (2016), pages 1309-1313, which is incorporated herein by reference. Under uniform magnetic fields, or field gradients, dipolar interactions between the MNPs favor magnetization along the chain direction and cause selective lifting.

Accordingly, there is a need for devices able to move in a viscoelastic media, by at least one applied stimulus field (SF).

### SUMMARY OF THE INVENTION

In one embodiment, this invention provides a device for implanting in a biological tissue and adapted to move in a viscoelastic media, the device comprising:
a main-body comprising a first material (M1) and having a direction in the viscoelastic media, and wherein the direction of the main body changes upon application of a first stimulus field (SF1) at a first threshold (T1); and
one or more memory shaped elements (MSE) having a first configuration and comprising a second material (M2), said second material comprises an elastomer, and wherein the MSE adopts a second configuration upon application of a second stimulus field (SF2) at a second threshold (T2).

In one embodiment, the second material (M2) is different from the first material (M2≠M1). In one embodiment, SF1 and SF2 are of the same nature (i.e., based on the same physical principle, for example, both fields are ultrasound (US) fields, magnetic fields, electric fields or electromagnetic fields) and the same direction; and wherein T2 is larger than T1. In one embodiment, the material of at least some of the MSEs are different one from another (M2ᵢ≠M2ⱼ, i≠j). In one embodiment, at least one of M1 and M2 comprises a form of micro- or nano-particles. In one embodiment, the first or second configuration of the MSE is selected from a group consisting of: an elongated shape, a film, a wire, a string, a strip, a plug, a sheet, a membrane, flagellum, coil, helix, arm, joint and any combination thereof. In one embodiment, at least one MSE is externally attached to the main-body, and adapted to propel the main-body in the viscoelastic media. In one embodiment, the application of the SF2 comprises cycles of the second stimulus field above and below the second threshold (T2).

In one embodiment, the main-body further comprises at least two fins, configured to steer the direction of the main-body. In one embodiment, the fins comprise the first material (M1). In one embodiment, the fins comprise a polarity direction at an angle relative to the main-body. In one embodiment, the fins are externally and symmetrically attached to the main-body. In one embodiment, the fins are configured to tilt relative to the main-body.

In one embodiment, the main-body further comprises a sealable cavity and when the MSE is in the first configuration the cavity is closed and in the second configuration the cavity is open. In one embodiment, the sealable cavity is configured to temporarily accommodate at least one of: a therapeutic entity, a therapeutic load, a diagnostic load, or a combination thereof. In one embodiment, the sealable cavity is configured to temporarily accommodate an explosion material, configured to propel the main-body.

In one embodiment, the device further comprising a sensitive sealing lid, configured to temporarily seal the cavity; wherein the sensitive sealing lid is configured to be opened responsive to an environmental threshold. In one embodiment, the MSE is configured as a sealing lid for the cavity; and wherein the configuration of the MSE opens and/or closes the sealable cavity. In one embodiment, the MSE comprises a first arm and pulls and/or pushes a sealing-lid of the cavity upon application of SF2. In one embodiment, the first arm comprises at least one element selected from: a spring, a helical spring, a leaf spring, a rod, a shaft, a pole and a bar. In one embodiment, the main-body further comprises a cavity and wherein the MSE comprises a second arm, configured to push a substance accommodated within the cavity out of the cavity upon application of SF2.

In one embodiment, this invention provides a system comprising:
a device described herein; and
a remote controlling module configured to control the application of SF1 and SF2.

In one embodiment, the remote controlling module comprises at least one inducer for a stimulus field selected from: magnetic, electric, acoustic, ultrasound, heat, X-ray, radio-wave and any combination thereof.

In one embodiment, the system further comprises a delivery and/or retraction module, configured to deliver and/or retract the device to and/or from a specific location selected from: in vitro, ex vivo, in vivo in a mammalian subject, and in vivo in a human patient. In one embodiment, the delivery and/or retraction module comprises an attachment element selected from: a magnetizable needle, expandable magnetic element, magnetizable surface, pneumatic element, electromagnetic element, ultrasonic element, deployable mesh, deployable micro-net, suction element, and any combination thereof. In one embodiment, the remote controlling module comprises a monitoring-device, configured to locate and display location and orientation of the device within the viscoelastic media.

In one embodiment, this invention provides a method comprising applying at least one of the stimulus fields (SF) to a device described herein to manipulate motion of the main-body within the viscoelastic fluid of a subject. In one embodiment, manipulation comprises: steering the main-body to a desired direction via an SF1 corresponding to the lower threshold (T1); and/or propelling the main-body by modifying the configuration of the MSE, via an SF2 corresponding to the second threshold (T2).

In one embodiment, the method further comprising at least one of:
externally loading the device's cavity with a selected load;
delivering the device into a treated subject;
monitoring the device's location and orientation within the viscoelastic media;
releasing the selected load from the cavity at a desired location;
imaging the subject to locate the device for further diagnostic information; or
retracting the device from a pre-determined location.

In one embodiment, the step of delivering comprises at least one of: injecting, providing for swallow, penetrating via catheter. In one embodiment, the step of releasing the selected load comprises modifying the configuration of the MSE via the SF2 at the second threshold (T2), such that the cavity's sealing lid is opened. In one embodiment, the step of releasing the selected load comprises opening the sensitive sealing lid, by providing a selected environmental threshold.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A, 1B and 1C schematically demonstrate a device having a flagellum, according to some embodiments of the invention;

FIGS. 2A and 2B schematically demonstrate a device having a cavity, according to some embodiments of the invention;

FIGS. 3A and 3B schematically demonstrate another device having a cavity, according to some embodiments of the invention;

FIGS. 4A and 4B schematically demonstrate another device having a cavity, according to some embodiments of the invention;

FIGS. 5A and 5B schematically demonstrate another device having a cavity, according to some embodiments of the invention;

FIG. 5C schematically demonstrates another device having a cavity, according to some embodiments of the invention;

FIGS. 6A, 6B, 6C, 6D, 6E and 6F schematically demonstrate a device with fins, according to some embodiments of the invention; and

FIG. 7 schematically demonstrates a system, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The term “device” herein denotes any object that is implantable in biological tissue. The terms “carrier device” and “carrier” herein denote a device that is capable of carrying and releasing a medical payload into the tissue. The term “medical payload”, or equivalently the terms “payload” and “cargo” used in a medical context is understood herein to include any substance or material, a combination of several relevant therapeutic materials, diagnostics or a combination of therapeutic and diagnostics. In certain embodiments of the invention, a fluid payload is used; the term “fluid” herein denotes that the payload is capable of flowing. In certain embodiments of the present invention, a solid payload is used; the term “solid” herein denotes that the payload can be released in the form of discrete particles. A device may be fabricated by known manufacturing techniques, including, but not limited to, 3D printing, molding, casting, etching, lithography, thin-film technologies, deposition technologies, and the like.

In various embodiments of the present invention, carrier devices are miniaturized for implantation in biological tissues. The term “miniaturized” (with reference to a device) herein denotes a device of small size, including, but not limited to: devices of millimeter to centimeter scale; devices of micrometer (“micron”) scale, referred to as “micro-devices”; devices of nanometer scale (including hundreds of nanometers), referred to as “nano-devices.” Not only are the devices themselves of the size scales as indicated above, but the devices' individual components are also of comparable scale.

According to some embodiments of the invention, a micro-/nano-device is provided comprising elastomer films with chained magnetic particles, which are configured for selective and directional actuation, for applications such as: propulsion, steering, and controlling the motion of the device. According to some embodiments, the elastomer films can control elements of the device such as open and/or close compartments thereof.

According to some embodiments, the diameter or actual length of the overall device is selected from: between 100 and 5,000 micrometers, between 10 and 100 micrometers, between 1 and 10 micrometers, between 200 and 1,000 nanometers, and any combination thereof. According to some embodiments, the diameter or actual length of the overall device is from 200 nanometers up to 5,000 micrometers.

A skilled artisan will appreciate that, memory shaped elements (MSEs), may refer according to some embodiments, to smart materials that are able to return from a deformed state (deformed under an applied stimulus field) to their original shape, when the stimulus field is removed or at least under (or alternatively above) a predefined threshold/s.

A skilled artisan will appreciate that, the phrase “applying the stimulus field corresponding to a threshold” or similar phrases may refer to applying the stimulus field such that it crosses a threshold (above or below, depending on the specific application), such that at least one material of the device reacts. For a non-limiting example, a thermal stimulus field can be applied where: for a heating stimulus a reaction occurs above a predetermined temperature (such as material melting) and for a cooling stimulus a reaction occurs below a predetermined temperature (such as material freezing).

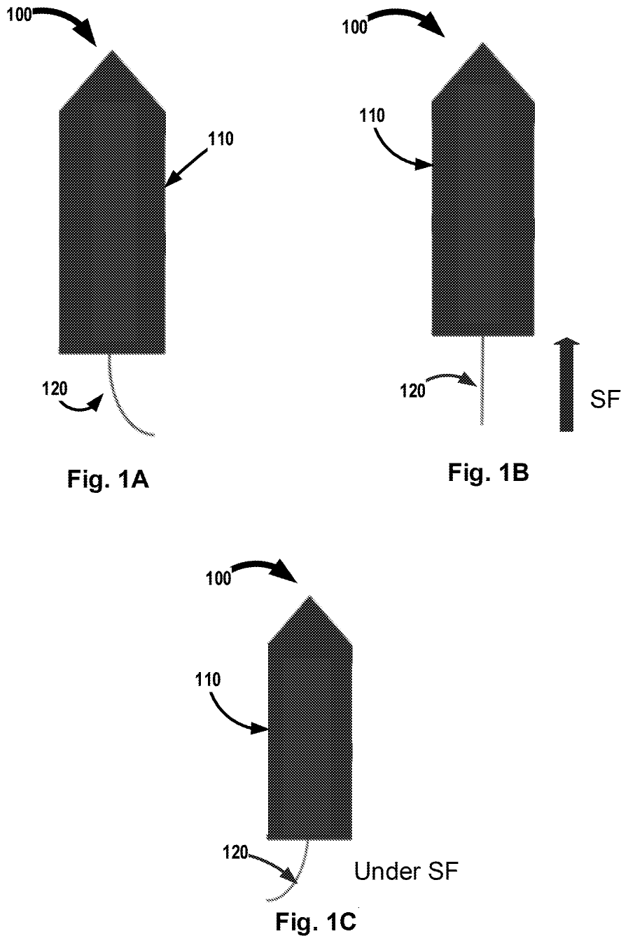

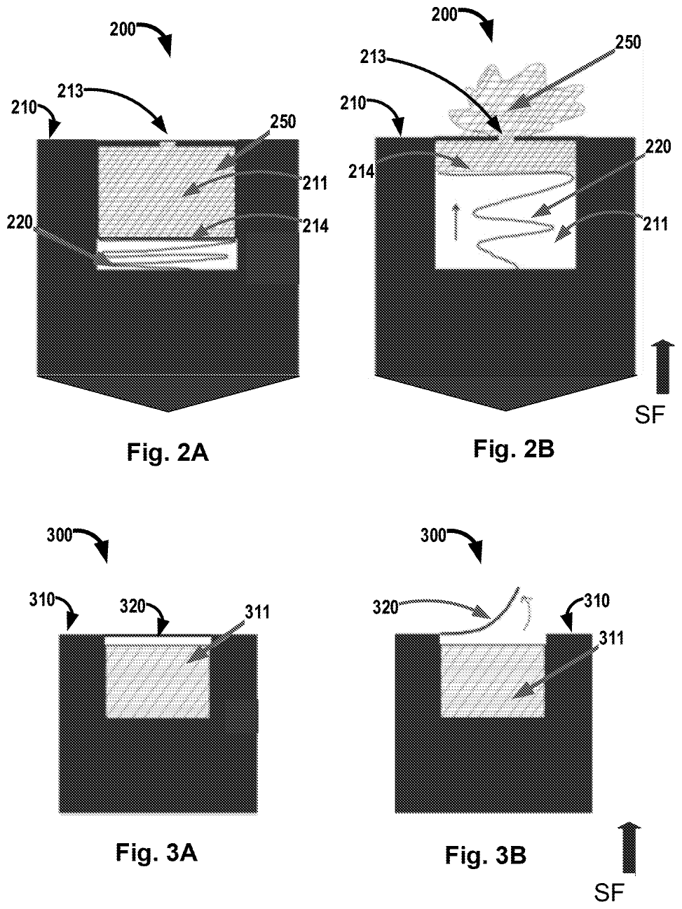

Reference is now made to FIGS. 1A-1C and 2A-2B. According to some embodiments, a device [100, 200] is provided and configured to move and travel in a viscoelastic media, responsive to an application of at least one stimulus field (SF); the device [100, 200] comprising:
a main-body [110, 210] comprising a first material (M1), M1 is configured to respond to an applied SF corresponding to (higher- or lower-than) a first threshold (T1); and
one or more memory shaped elements (MSEs) [120, 220] comprising a second material (M2), M2 is configured to deform responsive to an applied SF corresponding to (higher- or lower-than) a second threshold (T2);
wherein M1 is selected to enable manipulation of the main-body's direction in the viscoelastic media; and wherein M2 is selected to enable manipulation of MSE shape.

According to some embodiments, the MSE is configured to return to its original shape, once the SF is removed, or applied respectively (to the above mentioned) lower- or higher-than the second threshold. According to some embodiments, the SF is applied in a pulsatile (on/off) fashion.

According to some embodiments, the shape/s of the MSE is/are configured to propel the main-body in the viscoelastic media.

According to some embodiments, the second material is different from the first material (M2≠M1). According to some related embodiments, the materials M1 and M2 are both configured to react (respond/deform, respectively) to the same type of same SF. According to some related embodiments, the materials M1 and M2 are selected, such that upon the application of the SF, their corresponding first- and second-thresholds (T1≠T2) initially enable the activation of the first material (SF causing the main-body to respond) and then, with a higher SF application enable the activation of the second material (SF causing the MSE to deform); or vise-versa: initially activate the second material and then with a higher application of the SF activate the first material; depending on the selected application. Examples with the application of magnetic stimuli field are described in Examples 1 and 2.

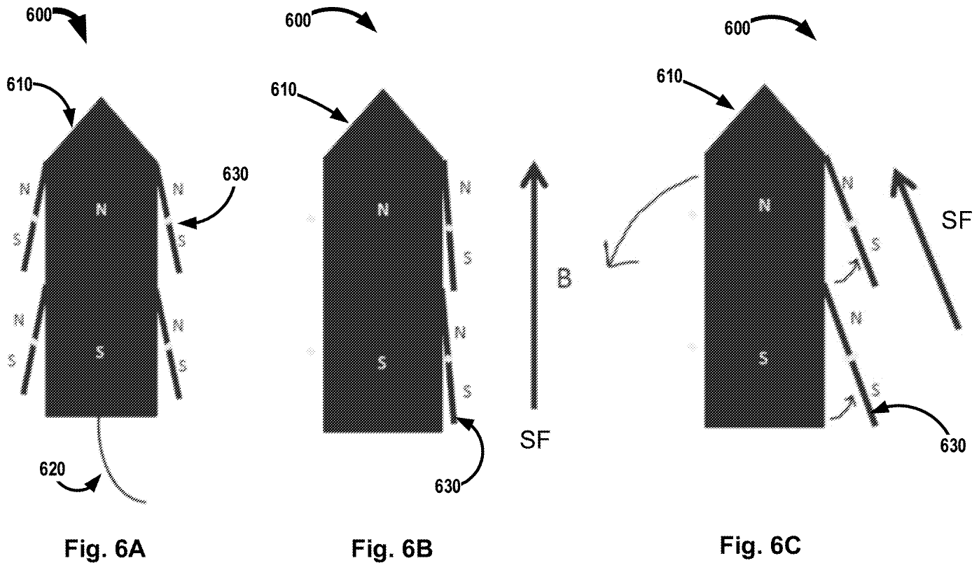

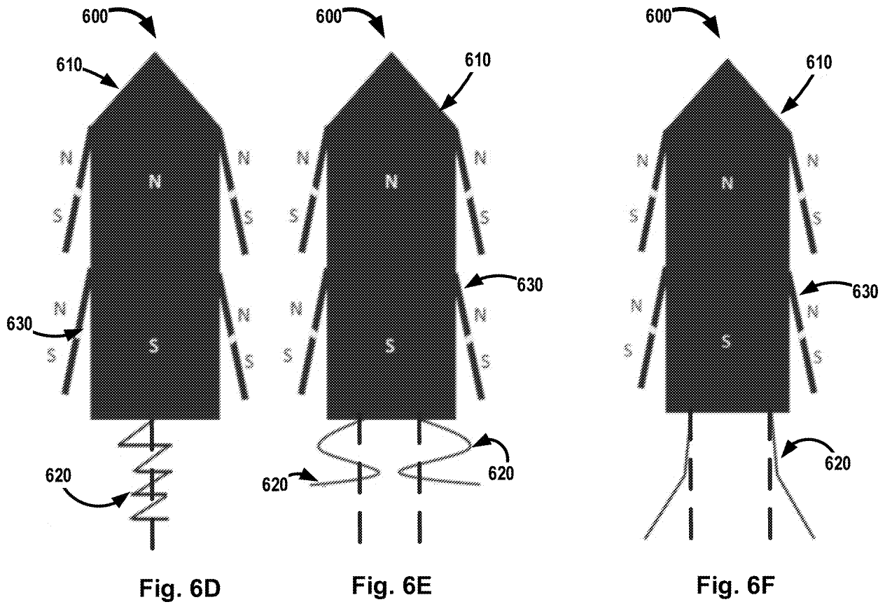

According to some embodiments, the second material (M2) is selected such that the applied SF (corresponding to the second threshold T2) is configured to deform the MSE and align its shape along the direction of the applied SF. FIG. 1A demonstrates an MSE [120] in its original shape, before application of the SF; and FIG. 1B, demonstrates the aligned MSE [120], during the application of the SF corresponding to the second threshold (T2). In FIGS. 1A-1C and also in FIGS. 6A-6E the MSEs [120,620] are designed as flagellum/flagella configured to propel the main-body in the viscoelastic media.

According to some embodiments, the second material (M2) is selected such that the applied SF (corresponding to the second threshold T2) is configured to deform the MSE into a predetermined shape (different from its original shape). FIG. 1A demonstrates the MSE [120] in its original shape (twisted to the right side), before the application the SF; and FIG. 1C, demonstrates the predetermined deformed shape MSE [120] (twisted to the left side), during the application of SF corresponding to a second threshold (T2). Another example is in FIG. 2A which demonstrates the MSE [220] in its original (compressed) shape, before the application the SF; and where FIG. 2B, demonstrates the predetermined deformed (expanded) shape MSE [220], during the application of SF corresponding to a second threshold (T2).

According to some embodiments, in the case of a plurality of MSEs, their materials M2 can be selected to be different, at least for some of the MSEs, or different per each MSE; namely selecting materials (M2₁, M2₂, . . . M2ₙ), such that each of the MSEs deforms under an applied SF corresponding to its respective second threshold (T2₁, T2₂, . . . T2ₙ).

According to some embodiments, the main-body comprises a shape selected from elongated, axisymmetric, centrosymmetric, chiral, random and any combination thereof.

According to some embodiments, the response of the main-body and/or sections thereof to the SF comprises at least one of: rotate, modify orientation, propel, oscillate, undulate, translate, expand, constrict, tilt away, tilt towards and a combination thereof.

According to some embodiments, the viscoelastic media comprises a material selected from: human blood, mammalian blood, biological tissue, biological organ and/or system, natural gel, synthetic gel, lymph, bile and a combination thereof.

According to some embodiments, the stimuli field is selected from: magnetic, electric, electro-magnetic, optical, acoustic, ultrasound, photoacoustic, radio waves, thermal, pH, solution, immunological, redox, thermal, enzymatic, protein, X-ray, cellular compartment-specific environment, and a combination thereof.

According to some embodiments, at least one of the stimuli fields is externally applied. According to some embodiments, at least one of the stimuli fields is internally applied. According to some related embodiments, the internally applied stimuli field is location related or dependent, namely depends upon the device's current location; for a non-limiting example, a pH level at a specific organ within a human (or other mammalian) body.

According to some embodiments, at least one of the first- and second-materials comprises a form of micro- or nano-particles.

According to some embodiments, at least one MSE comprises an elastomer material (as mentioned in the background) having a configuration selected from a group of: an elongated shape, a film, a wire, a string, a strip, a sheet, a plug, a membrane, flagellum, coil, helix, arm, joint and any combination thereof. Embodiments disclosed herein for an elastomer film also apply to other configurations from the list presented herein above.

According to some embodiments, at least one MSE comprises a material selected from: composite memory polymer that contains embedded electric, magnetic-sensitive material, acoustic-sensitive material, microwires, diverse microparticles, microirregularities, layered 2D/3D nano-/microstructures, pH-sensitive material, redox-sensitive material, specific enzyme-sensitive coating that triggers reversible or irreversible topological change, and any combination thereof.

According to some embodiments, and as demonstrated at least in FIGS. 1A-1C, at least one MSE [110] is externally attached to the main-body (for example a flagellum [120]), configured to propel the main-body in the viscoelastic media, responsive to the application of the SF corresponding to the second threshold (T2). According to some related embodiments, the SF application comprises cycles of the SF above—and below—the second threshold (T2). According to some related embodiments, the cycles of application can be a frequency application of the stimuli field.

According to some embodiments, and as demonstrated in FIGS. 6A-6C, the main-body [600] further comprises at least one fin [630], configured to steer the direction of the main-body. According to some embodiments, the fins are configured to tilt relative to the main-body [610], thereby rotate, propel and/or turn the main-body within the viscoelastic media, as demonstrated in FIGS. 6A-6C: before the application of the SF (as in FIG. 6A), and during the application of the SF corresponding to a first threshold (T1), as in FIGS. 6B and 6C for different directions of the SF.

According to some embodiments, the fins are smaller than the main-body. According to some embodiments, the fins are positioned in an axisymmetric arrangement. According to some embodiments, at least one of the fins is flexible. According to some embodiments, at least one of the fins is rigid. According to some embodiments, the fins are attached to the main body by pins and/or joints. According to some embodiments, the fins are attached to the main body via adhesive elements or methods.

According to some embodiments, the fins [630] comprise a third material (M3). According to some embodiments, materials M1 and M3 both configured to react to the same SF. According to some embodiments, the fins comprise the first material (M3=M1). According to some embodiments, the fins have the same fixed polarity direction as the main-body. For example, and as demonstrated in FIGS. 6A-6F, the direction of magnetization polarity (or alternative force field vector) is parallel or slightly tilted relative to the axis of symmetry of the main-body [610].

According to some embodiments, and as demonstrated for devices [200, 300, 400, 500, 800] in FIGS. 2A-2B, 3A-3B, 4A-4B, 5A-5B and 5C the main-body [210, 310, 410, 510, 560] further comprises a sealable cavity [211,311,411,511,561]. According to some embodiments, the volume of the cavity is selected from between 5% and 95% of the main-body. According to some embodiments, the volume of the cavity is selected from 5%, 10%, 15%, 20%, 25%, 30%, 35%, 40%, 45%, 50%, 55%, 60%, 65%, 70%, 75%, 80%, 85%, 90% or 95% of the volume of the main-body.

According to some related embodiments, the sealable cavity is configured to temporarily accommodate a predetermined load selected from at least one of: jet load, diagnostic load, therapeutic load, therapeutic entity and a combination thereof. According to some embodiments, the sealable cavity is configured to temporarily accommodate multiple therapeutic entities and multiple diagnostic loads in predetermined combination thereof.

According to some embodiments, the jet material is remotely activated and the jet's torque is configured to propel the main-body in the viscoelastic material. According to some embodiments, the application of the SF at a predetermined level activates the jet propulsion-generating material.

According to some related embodiments, the MSE [320, 420, 520, 562] is configured to control the opening and closing of the cavity, responsive to the application of the SF corresponding (above- or below-) to the second threshold (T2); for example, by opening a sealing lid [320,412,512,562] of the cavity, as respectively shown in FIGS. 3B, 4B, 5B and 5C.

According to some related embodiments, the MSE [220,320,420,520,562] is configured to release a selected load accommodated within the cavity [211, 311, 411, 511, 561], responsive to the application of the SF corresponding (above- or below-) to the second threshold (T2). For example, by pushing or extruding the load via a small opening hole of the cavity [213], as demonstrate in FIG. 2B, or by opening the cavity as demonstrated in FIGS. 3B, 4B, 5B and 5C.

According to some related embodiments, therapeutic entities can be loaded into the cavity, and comprise at least one of: radionuclides, alpha-particles and neutron emitters, small peptides, peptoids, antibodies, antibody-drug conjugates, modified antibodies and their derivatives as exemplified but not limited to light chain antibody constructs, nucleic acids as exemplified but not limited to aptamers, antisense oligonucleotides, RNAi, siRNAs, shRNAs, miRNAs.

In some embodiments, the therapeutic load can comprise components of CRISPR-Cas9 or related gene editing molecules. In some embodiments, the therapeutic load can include vaccines as exemplified but not limited to the Bacillus Calmette-Guerin vaccine. In some embodiments, the therapeutic load can include oncolytic viruses as exemplified but not limited to Talimogene laherparepvec (OncoVEX GM-CSF). In some embodiments, the therapeutic load can include specialized cells and or cell therapy as exemplified by but not limited to CART cells or pluripotent stem cells. In some embodiments, the load can include diagnostics and contrasting agents including but not limited to radio-, MRI- or ultrasound contrast agents. In some embodiments, the cavity described therein can contain active agents as solids, solutions or alternative formulations including gels, sols, suspensions, nano- or microformulations of therapeutic agents including but not limited to micelles, liposomes, mesoporous silica-, carbon nanotube-mediated carriers their composites or alternative particles that supply intended therapeutic load of an agent or their mixtures and fit the cavity.

According to some embodiments, and as can be seen from FIGS. 2A-2B and 3A-3B (but not limited to), the sealable cavity [211,311] is configured to temporarily accommodate an expulsion material, configured to propel the main-body [210,310]. According to some embodiments, the expulsion material is configured to be triggered by a predetermined threshold to the applied SF.

According to some embodiments, the device further comprises a sensitive sealing lid, configured to temporarily seal the cavity. The sensitive sealing lid is configured to be opened (for example dissolve, melt, bend) responsive to a threshold to an environmental local field (not by the applied SF) selected from: acoustic, ultrasound, temperature, pH, redox, enzymatic, protein, cellular compartment.

According to some embodiments, and as demonstrated in FIGS. 3A-3B and 5C, the MSE is configured as a sealing lid [320,562] for the cavity [311,561]; and wherein manipulation of the MSE's shape is configured to open and/or close the cavity.

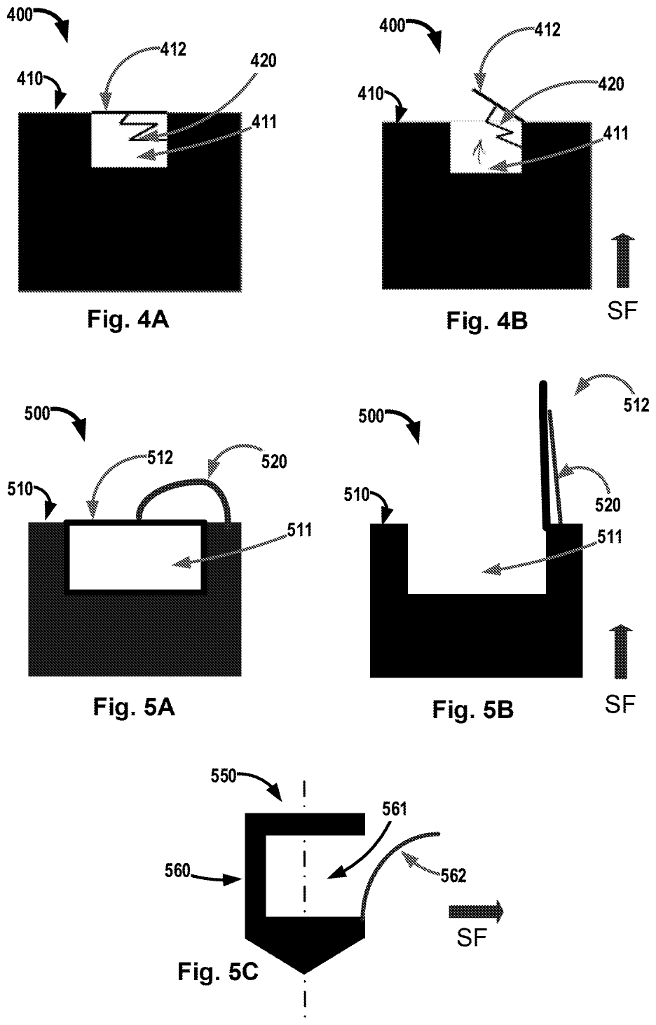

According to some embodiments, and as illustrated in FIGS. 4A-4B and 5A-5B, the MSE is configured as a first arm [420,520], configured to pull and/or push a sealing-lid [412,512] of the cavity. FIGS. 4A-4B illustrate the first arm [420], configured to open/close the sealing lid [412] from within the cavity [411], while FIGS. 5A-5B illustrate the first arm [520], configured to open/close the sealing lid [512] from an external side of the cavity [511].

According to some embodiments, and as demonstrated in FIGS. 2A-2B, the MSE is configured as a second arm [220], configured to push a tray [214] on which the load is accommodated, and thereby to push that load out of the cavity [211], responsive to the application of the SF corresponding to a second threshold (T2).

According to some embodiments, at least one of the first- and second-arms is selected from: a spring, a helical spring, a leaf spring, a rod, a shaft, a pole, and a bar.

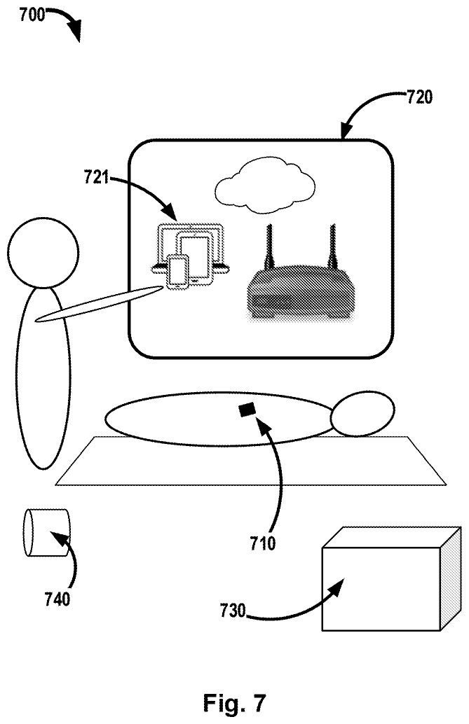

According to some embodiments of the invention, and as demonstrated in FIG. 7, a system [700] is provided comprising:
At least one device [710] of one of the above-mentioned embodiments [100, 200, 300, 400, 500, 600, 550]; and
a remote controlling module [720] to control the application of the stimuli fields (SF), thereby manipulating the direction of the main-body in the viscoelastic media and to the shape of the MSE.

According to some embodiments, materials of one device are different from another, accordingly their corresponding thresholds.

According to some embodiments, the remote controlling module [720] comprises a monitoring device [721], configured to locate and display the location and orientation of the device [710] within the viscoelastic media.

According to some embodiments, the remote controlling module [720] comprises an input device [721] to be handled by a caregiver, configured to provide instructions to the device's [710] motion within the viscoelastic media.

According to some embodiments, the remote controlling module [720] comprises at least one inducer [730] for a stimulus field selected from: magnetic, electric, piezoelectric, acoustic, ultrasound, heat, X-ray, radio-wave, optical and any combination thereof.

According to some embodiments, the magnetic field inducer [730] comprises a set of permanent magnets and/or conducting coils (such as Helmholtz or Maxwell coils) generating an arbitrary magnetic field vector at predefined location, where the main-body and MSE are located. Such magnetic field vector can be adjusted to control direction of the main body and shape of the MSE. According to some embodiments, a combination of coils and/or fixed magnets can generate the magnetic field.

According to some embodiments, the remote controlling module [720] is configured to control features of the SF selected from: power, intensity, frequency and direction; for a non-limiting example: to focus an ultrasound via a series of diverse transducers to adjust to a specific topology and depth. According to some embodiments, the remote controlling module [720] is configured to control a combination of aforementioned external stimuli to control both the main body and MSE in a synergistic or discrete fashion; for a non-limiting example, using electromagnetic and ultrasound stimuli to remotely control specific aspects of the device's [710] propulsion.

According to some embodiments, the system [700] further comprises a delivery and/or retraction module [740], configured to deliver and/or retract the device to—and/or from—a specific location selected from: in vitro, ex vivo, in vivo in a mammal, or in vivo in a human patient. According to some embodiments, the module comprises an attachment element selected from: magnetizable needle, pneumatic element, expendable magnetic element, magnetic surface, electromagnetic element, ultrasonic element, deployable mesh, deployable micro-net, suction element, and a combination thereof.

According to some embodiments, the delivery and retraction module is aimed at controlled delivery and collection of nano- or micro-devices to and from a specific location prior to and after actuation with external stimuli and cargo delivery. According to some embodiments, the module can comprise one or several structural elements to deliver and collect said nano- or micro-devices. According to some embodiments, the module can contain specific design to secure single or multiple insertions for in vitro, in vivo or patient applications. According to some embodiments, the module can contain a magnetic or magnetizable needle for injecting and collecting the nanos or micro-devices. According to some embodiments, the module can contain alternative delivery techniques based on electromagnetic, ultrasound or pneumatics-based devices. According to some embodiments, the module can contain alternative collection techniques as exemplified but not limited to deployable mesh, micro-net or suction. According to some embodiments, the magnetic needle can be designed to accommodate a standalone device or a device in a matrix to secure precise delivery. According to some embodiments, the magnetic or magnetizable needle can be kept in the injection matrix in vitro, in vivo or in patient for the duration of treatment or retracted and reintroduced for device collection.

According to some embodiments of the invention, a method of use is provided, to treat and/or monitor (for example, delivering a therapeutic entity) a desired tissue or subject selected from: in vitro, ex vivo, in vivo system of the subject (e.g., a mammalian body or a patient), using the device and/or system of the above-mentioned embodiments. The method comprising: applying at least one of stimulus field (SF) configured for manipulating motion of the main-body within the viscoelastic fluid of the subject.

According to some embodiments the step of manipulating comprises: steering the main-body to a desired direction via an SF corresponding to the threshold (T1); and/or propelling the main-body by modifying the shape of the MSE, via an SF corresponding to the second threshold (T2).

According to some embodiments, the method further comprises at least one of (not necessarily in that order):
externally loading the device's cavity with a selected load;
inserting and/or delivering the device into a treated subject;
monitoring location and orientation of the device within the viscoelastic media;
once required, releasing the selected load or therapeutic entity from the cavity, thereby at the desired location;
imaging the subject, for locating the device, or for further diagnostic information;
collecting and/or retracting the device (optionally after treatment) from a pre-determined location.

According to some embodiments, the step of inserting comprises at least one of: injecting, piercing, inserting, prying, providing for swallow, penetrating via catheter.

According to some embodiments, the step of releasing the therapeutic entity comprises modifying the shape of the MSE via an SF that corresponds to the second threshold (T2), such that the cavity's sealing lid is opened.

According to some embodiments, the step of releasing the therapeutic entity comprises opening the sensitive sealing lid, by providing a selected environmental threshold.

According to some embodiments, opening of the sensitive sealing lid can be provided by a tunable ultrasound of particular power in the range of 10-200 Watt, with an intensity in the range of 0.01-1.0 Watt/cm², a diverse pulse ratio as exemplified but not limited to 1:4/3 (20%, 25%) or 1:1/Continuous (50%, 100%), and frequencies in the range of 10-60 KHz or 0.25-30.0 MHz.

According to some embodiments, opening the sensitive sealing lid can be provided by a tunable pH sensitive membrane that undergoes open-close-open transition(s) in the range of 3-8 as exemplified by but not limited to hydrazones, Schiff bases (imines), trityl groups, acetals/ketals, oximes, 1,3,5-triazaadamantanes, and boronate esters.

According to some embodiments, opening the sensitive sealing lid is provided by a tunable thermo-sensitive membrane that undergoes open-close-open transition(s) when exposed to local gradients of thermal changes, when treated with external stimuli as exemplified by but not limited to magnetic, electric, acoustic or (ultra) short wavelength light fields. According to some embodiments, the lid undergoes a conformational thermally-induced open-close-open transition in the interval of 37-80° C. According to some embodiments, the diameter or actual length of the overall device is selected from: between 100 to 5,000 micrometers, between 10-100 micrometers, between 100 nanometers and 10 micrometers, and any combination thereof as determined at the surface. The conformation change can be reversible, partially reversible or irreversible to mediate multiple steps or a single step release of a therapeutic load as exemplified by a membrane that exhibits a proper chemical moiety that undergoes a chain-ring transformation upon thermal exposure as exemplified by lactams and lactones. The external field can be applied continuously or in controlled pulses to maintain proper release vs. safety ratio.

According to some embodiments, opening the sensitive sealing is provided by a tunable redox-sensitive membrane that undergoes open-close-open transition(s), when exposed to concentration gradients for media-specific molecules as exemplified but not limited to arylboronic acids, thioketals, disulfide bridges or specific biological molecules that contain thereof, including but not limited to dithiothreitol, glutathione, cysteine- or methionine-containing peptides and proteins.

According to some embodiments, opening the sensitive sealing lid is provided by a tunable enzyme- or other biological molecule-sensitive membrane that undergoes open-close-open transition(s), when exposed to concentration gradients for media-specific molecules. According to some embodiments, the sealing lid may contain peptidic sequences sensitive to local gradients of phosphatases (for linkers with cleavable phosphate groups), esterases for the degradation of ester bonds, glycosidases, and proteases that cleave specific oligopeptides (e.g., GlyPhe-LeuGly).

### Example 1

An example with a magnetic stimuli field is provided, according to some embodiments of the invention. In this example, a steering and propulsion device is provided to move or travel in a viscoelastic media on the nano-/micro-/milli-meter scale, using external magnetic fields. The materials of the device include a combination of elastomer-based flagellum for propulsion and a magnet-based main-body and fins for directional steering. Such a device can be used to propel a particle inside a human body via the tissue, carry medical payloads (therapeutics or diagnostics) or conduct minimally invasive surgery.

As shown in FIGS. 6D-6E, particle (device [600]) comprises three main components:
the main-body [610] of the magnetic particle, with a fixed polarity, corresponding to the desired direction of motion, based on an embedded magnetic component with a sufficiently strong magnetic moment;
smaller magnetic fins [630] attached to the main-body symmetrically all around its axis (cylindrical symmetry); where each fin has a fixed polarity, aligned with the polarity of the main-body of the particle, based on an embedded magnetic component in the fin with a sufficiently strong magnetic moment. Such fins can be produced, for example, from the elastomer films that are described in Mishra et al., or alternatively by other suitable techniques known in the art. Such films comprise for example Fe₃O₄ magnetic nanoparticles (MNPs) and thermoplastic polyurethane (TPU). The films are nanocomposites comprising the polymer and the magnetic nanoparticles. Assembly of the MNPs into chains causes a directional dependence in the magnetostatic energy, allowing for anisotropic actuation of the composite in 3D. The fins are attached to the main-body with a flexible attachment point and/or are made of flexible material, so they can tilt or “flap” when placed in an external magnetic field (since they are magnetic, they can tilt to align with the external magnetic field); in this case the main-body of the particle is also subject to a rotating torque aligning it with the external magnetic field; since the body is larger than the fins it can tilt more slowly, subject to drag in viscoelastic media, allowing the fins to “flap” relative to the body; and
a flagellum (or multiple flagella as in FIG. 6E), at the tail end of the main-body, made of elastomer with embedded magnetic nanoparticles (MNPs).

In this example, the MNPs in the flagella are based on a magnetic material M2, which is different (in terms of magnetic permeability, magnetic moment) from the magnetic material used for the main-body and fins (M1, M1′ respectively); the reason for such material selections are as follows.

Particle motion is controlled by an external magnetic field:

B=B1+B2, Eq. 1
where:
   B1 is a fixed low amplitude (low power) steering component (changing direction only when the particle is required to turn), and
   B2 is a varying amplitude, high power, on-off pulse component, which is in charge of propulsion;
both B1 and B2 vectors are in the same direction.

When B=B1 (meaning B2=0) the flagella remain in their relaxed position, since the flagella are based on elastomers with embedded magnetic material M2, with a magnetic moment that is too weak to generate sufficient torque for flagella movement under field B1.

In contrast, the materials M1, M1′ used respectively for the particle body and fins have a magnetic moment large enough to generate rotational (steering) particle movement under field B1.

Importantly, material M2 does not necessarily have a lower magnetic moment compared to M1, M1′ per unit volume or mass. However, M2's magnetic moment is too weak relative to the minimal threshold needed for flagella activation (i.e., field B1 generates torque strong enough to steer the main-body and fins, but not strong enough to activate the flagella). The minimal threshold (T2) to activate the flagella depends on elastomer mechanical characteristics, such as dynamic moduli, flagella geometry and size, as well as surrounding medium rheology. The minimal threshold (T1) to steer the main-body and fins depends on the surrounding medium rheology, as well as particle geometry and size.

In summary, the flagella do not change their shape under the weak magnetic field B1. Only when B is clearly greater than B1 (i.e., B2>>0) the external field is high enough to activate the flagella and make them change their shape. The on-off changes in flagella shape as a result from the on-off pulses of B2 generate the motion of flagella that propels the particle forward.

FIGS. 6E and 6F demonstrate a configuration where the flagellum has two possible configurations of minimal potential (symmetrical to each other). In each of those configurations the flagellum is curved, either to one side or to the other. When a strong external magnetic field B is switched on, the flagellum straightens (marked with dashed lines), reaching a potential local minimum point (in the middle between the two symmetrical global potential minima points). This configuration is referred to as a bi-stable structure, supported by two orthogonal curvature axes (parallel to the two sides of the rectangular elastomer sheet). An example of such a structure is a “snap bracelet”. When the external magnetic field is switched off, the flagellum snaps back to either one of the potential minima points (with equal probability). When the field B2 component is repeatedly switched on-off, this on average results in a flip-flop motion between the two potential minima configurations of the flagellum (analogously to a fish tail fin motion), thus propelling the particle (device [610]) forward. According to some embodiments, when field component B2 is kept switched off, the flagellum rests in one of the two stable potential minima configurations (not flip-flopping). Only when the B2 component is switched on, the flagellum arrives at the unstable middle position, from which it will randomly flip to one of the two stable positions, once the field component B2 is switched off.

FIG. 6E illustrates a configuration where there are two flagella, which have symmetrical curved shapes when there is no strong external magnetic field B2 (similar to a frog's legs). When B2 is large, the flagella straighten (marked with dashed lines), pushing the particle forward.

FIG. 6D shows a configuration where the flagellum in its relaxed position (without strong external field B2) has a folded accordion shape. When external field B2 is switched on, the flagellum straightens (marked with dashed line), pushing the particle forward.

According to some embodiments, each flagellum comprises an elastomer sheet with a particular shape (in three-dimension). To clarify, FIG. 6A-6F show cross-sections of the particles and their flagella, rendering each flagellum as a two-dimensional curve. Many other flagella configurations are possible, resulting in propulsion of the particle forward.

According to some embodiments, when B changes its direction the main-body and fins tilt to align with the direction of B, steering the particle in the desired direction; as shown in FIGS. 6A-6C (before SF application (6A) and for two different SF directions (6B and 6C)).

The combination between the steering component and the on-off propulsion pulse component is configured to generate a directed and accurate remotely-controlled motion of the device [600] in viscoelastic media.

According to some embodiments, the external magnetic field can be generated by permanent magnets, Helmholtz, Maxwell coils or a combination thereof around the target area (the current location of the device). The exact shape and size of fins, particles and flagella can be optimized to improve mobility in specific viscoelastic media.

The strength of the relevant magnetic fields B1, B2 can range anywhere between single-digit Gauss to single-digit Tesla (depending on particle size and geometry, materials used, and rheology of the medium in which the particle is moving). The sizes of the particles, fins and flagella can range between 10's of nanometers to 1-10 millimeters in any dimension.

Examples of magnetic materials M1, M1′, M2 that can be used include: iron, nickel, permalloy, cobalt, and others. For example, one may choose permalloy for the high permeability material and nickel for the lower permeability material, to ensure the flagella are not activated by the weak magnetic field B1 while the main-body/fins are affected by this field.

Approximated relationship between B1, B2, magnetic moments of materials M1, M1′, M2 is provided, according to some embodiments of the invention. The relationship between B1, B2 and the permeabilities of materials M1, M1′, M2 can be approximately described as follows.

Assuming that B1 has to be high enough to a generate torque t1 strong enough to cause particle [600] rotation, i.e. greater than a certain threshold (t1>T1) dependent on the rheology of surrounding medium and on particle size and shape.

t1 is approximated as: t1=C*B1*Mt, Eq. 2
where
   Mt is the total magnetic moment of the particle; Mt can be approximated as a linear combination of M1 (magnetic moment of the main-body) and M1′ (magnetic moment of the fins); this is the effective magnetic moment of the main-body and fins;
   the scale factor C depends on the angle between the external field and the particle axis (among other factors).

In order to trigger a rotation:

C*B1*Mt>T1 Eq. 3

Assuming B1+B2 are required to generate a torque t2 on the elastomer flagella, which is large enough to trigger a change in flagella shape. t2 is approximated as:

t2=A*(B1+B2)*M2 Eq. 4
the scale factor A is dependent on the flagella shape and angle in relation to the external magnetic field, among other factors.

In order to trigger a change in flagella shape:

A*(B1+B2)*M2>T2 Eq. 5
where T2 is the threshold torque required for shape change, dependent on elastomer properties and environment rheology.

Let's denote: B2=B1*N Eq. 6
where, N is a scale factor.

So, A*(N+1)*B1*M2>T2 Eq. 7

However, it is also required that: A*(B1)*M2<T2 Eq. 8
i.e., the flagella do not get activated without the large field component B2.

Let's assume that: T1=T2*D Eq. 9
where D is a scale factor.

Then by combining Eq. 3 and Eq. 8 one gets:

C*B1*Mt>T2*D>A*B1*M2*D Eq. 10

and therefore, C/(AD)>M2/Mt Eq. 11

This means that as long as M2 is not too high relative to Mt, the flagella cannot be activated by the field B1 alone.

A practical example for these measures. Assuming C=1 (equivalent to embedding the scale factor C in Mt), A=1 (equivalent to embedding scale factor A in M2). T1, T2, D are a given (i.e., physical parameters imposed on us). Mt, M2, B1, B2 are parameters one can choose.

Assuming one chooses a material generating total magnetic moment Mt for the particle main-body and fins. Denote Y=B1*Mt/T1. Since Mt, T1 have already been defined or chosen, one can now choose B1=(T1*Y)/Mt, to satisfy Y>1, so Eq. 3 is satisfied.

By choosing N to be D+1, so B2=(D+1)*B1. If one can choose magnetic material M2 so that M2=Mt/(Y*D), where Y>1, then Eq. 11 and Eq. 8 are satisfied, substituting into Eq. 5 so it is satisfied:

$$M 2 * ( D + 1 ) B 1 = ( Mt \text{/} ( YD ) ) * ( D + 1 ) B 1 = Mt \text{/} ( YD ) * ( D + 1 ) * Y T 1 \text{/} Mt = ( T 1 + T 1 \text{/} D ) > T 2 = T 1 \text{/} D \qquad Eq . 12$$

In other terms, one needs to choose M2 and Mt so that M2/Mt scales inversely with D=T1/T2. Since M2 and Mt scale with the respective materials' magnetic permeability, M2 and Mt can be set to meet the above criteria by appropriate choice of materials.

If D (i.e. T1/T2) ranges between 1/100 and 100 (a wide range encompassing nearly all practical ratios in a physical scenario), one needs to choose a pair of materials M2, Mt whose permeability ratio scales inversely (between 100 and 1/100). Multiple examples of such materials exist with a wide range of permeability ratios (such as nickel vs. permalloy) to readily select suitable materials for a desired ratio. If D≥1 then one chooses Mt to be based on the higher permeability material, and M2 to be based on the lower permeability material. If D<1 then M2 is based on the higher permeability material, and Mt is based on the lower permeability material.

### Example 2

According to some embodiments, a system is provided configured to release payloads (e.g., drug, therapeutic entities) encapsulated in a particle using an external magnetic signal, and based on a combination of elastomer-based membranes that are used to contain/release the payload.

The particle [200,300,400,500,550] as shown in FIGS. 2A-2B, 3A-3B, 4A-4B, 5A-5B and 5C is comprised of:
the main-body [210,310,410,510,560] of the particle, with a cavity [211,311,411,511,561] configured for containing the payload.
a membrane [220,320,420,520,562] made of an elastomer with embedded MNPs; the membrane can be attached to the cavity bottom in a spring-like fashion [220] (as illustrated in FIGS. 2A-2B), configured to push a tray [214] on which the payload is accommodated; the membrane can be used to as a lid [320] configured to seal the cavity and prevent free payload diffusion (as illustrated in FIGS. 3A-3B and 5C); or the membrane can be designed as an arm [420,520] (as illustrated in FIGS. 4A-4B and 5A-5B), configured to open and close a sealing lid [412,512] of the cavity.

According to some embodiments, when there is no external magnetic field the membrane is in its default relaxed position, preventing (or at least not facilitating) payload diffusion out of the particle (meaning out of the cavity).

According to some embodiments, when a specific external magnetic field is applied, the membrane either:
pushes the payload out of the cavity (as in FIG. 2B),
folds to open the cavity and allow diffusion (as in FIGS. 3B and 5C), or
pushes/pulls the sealing lid to open cavity (as in FIGS. 4B and 5B).

According to some embodiments, the several setups can be combined; i.e., two membranes—one opening/closing the cavity and the other pushing the payload out.

According to some embodiments, the device can be used in combination with magnetic particles (carrying the payload), which are propelled in viscoelastic media using an external rotating electromagnetic field. In this case, the entire particle is configured to rotate around its axis under the influence of the external rotating magnetic field. The plane of field rotation is orthogonal to the direction of motion. This rotation propels the particle forward like a corkscrew. According to some embodiments, inverting the direction of rotation of magnetic field propels the particle backwards, respectively.

The challenge is to ensure such a rotating external magnetic field does not activate the payload release mechanism described above. The solution: the particle body contains magnetic material M1. In contrast, the material of the elastomer membranes involved in the payload release mechanism comprise the embedded MNPs of magnetic material M2.

The external magnetic field has two components:

B=B1+B2 Eq. 13
where B1 is the steering and propulsion rotating magnetic field component.
The goal is to prevent this component from activating the drug release elastomer membranes when B2=0.

Three methods are provided to prevent this, which can be applied individually or in combination:

The exact direction of the field B2, required to activate the elastomer membranes, can be accurately designed (as part of the elastomer membrane design and its position on the particle [550]). In an example of an elastomer [562] design as in FIG. 5C, where the planar elastomer membrane only changes its configuration when the vector of external magnetic field is not parallel to the two-dimensional plane of the membrane [562]. Accordingly, when the membrane [562] is positioned on the particle [550] so that it is orthogonal to the particle axis of rotation (i.e., parallel to the plane of the external rotating magnetic field), then as long as there is no sizeable vector component of B in the direction of main-body [560] motion, the elastomer membranes are not activated, and the payload is not released.

According to some embodiments, one can design the particle (i.e., choose the materials M1, M2) so it is capable of propulsion by B1 of low amplitude. In this design, the magnetic elastomer is not activated under field B1 due to the magnetic moment of material M2, which is low compared to the minimal torque required for elastomer activation, while the particle main-body keeps rotating with the field B1, due to the magnetic moment of material M1 (which is high enough compared to the minimal torque required for particle rotation). Only when B2>>0 and B is substantially greater than B1, the magnetic elastomer in the membranes is activated and triggers payload release on demand. This can be done by appropriate choice of materials M1, M2 and fields B1, B2, as described in Example 1 above.

According to some embodiments, when magnetic field B1 rotates within a predefined operational plane and/or volume, which may be located inside a patient body, at a certain frequency F1, material M2 can be chosen by design such that, it responds to changes in an external magnetic field more slowly than the frequency F1 of the rotating field B1 (i.e., greater magnetic viscosity).

This choice can be combined with a specific membrane design that requires more time to change its shape in response to the change in external magnetic field. For example, when properly positioned in reference to the elastomer membrane (e.g., orthogonally to the membrane plane), the external field may exert aggregate torque t1 on the elastomer membrane (net of internal resistive forces in response to the shape deformation, which depend on the dynamic moduli of the elastomer membrane). The membrane starts deforming from a stationary position. It takes a minimal time x for the membrane to reach its fully extended position, which will allow payload diffusion. However, if the rotation frequency of the external field is high enough, then within time <<x the external magnetic field has rotated to a new angle relative to the membrane, at which the field no longer activates the membrane as the field component orthogonal to the membrane plane is lower than the threshold torque necessary for membrane activation. Therefore, the membrane never reaches its fully activated state. That means that as long as the external field is rotating, the elastomer never “catches up” with it, so it is not activated, and the payload is not released. A long fixed pulse B2 is activated only at the desired moment of payload release. This pulse is long enough to cross the threshold of the response time x for the elastomer membrane. Therefore, the elastomer membrane is activated, and the payload is released on demand.

According to some embodiments, all three of the above options can be combined by using a rectangular, double exponential, damped sinewave pulse or a combination thereof, within a range of 10 millisecond to 1 minute pulse of a high magnetic field in a direction orthogonal to the plane of the rotating low magnetic field.

The strength of the relevant magnetic fields B1, B2 can range anywhere between single-digit Gauss to single-digit Tesla (depending on particle size and geometry, materials used, rheology of medium in which particle is moving).

The size of the particles can range between 10's of nanometers to 10's of mm's in any dimension.

Examples of magnetic materials M1, M2 defined above that can be used include iron, nickel, permalloy, cobalt, and others. For example, one may choose permalloy for higher permeability and nickel for lower permeability to make sure the membrane is not activated by the weak magnetic field B1, while the body is affected by this field.

### Examples for Manufacturing Thin Elastomer Layers

According to some embodiments of the invention, manufacturing methods are provided for elastomer-based membranes and magnetic particles.

The motility appendages described above (various flagella as in Example 1) as well as the payload release control membranes/springs described above (as in Example 2) include, but are not limited to, magnetic polymer composites comprising a base polymer and a dispersed magnetic phase.

For example, flagella for the device can be manufactured via a template-based or template-free magnetic assembly. Specifically, the ‘frog legs’, accordion, or ‘fin’-shaped flagella can be manufactured using casting and/or molding techniques.

In a representative procedure, a preformed mold and/or cast is filled with a solution or neat liquefied polymer of choice (ex., polydimethylsiloxane) followed by addition of magnetic micro/nanoparticles to create a suspension.

The resulting suspension is allowed to cure in the presence of an external magnetic field or alternative source of energy (ex., ultrasound) in order to ascertain unified and/or patterned particle distribution throughout the polymer to furnish in the targeted magnetoactive elastomer material.

The resulting flagella can have ‘shape-memory’ features (“Stimulus responsive shape-memory materials: A review,” Materials and Design 33 (2012), pages 577-640) and be capable of being propelled by external magnetic field(s) as exemplified in FIGS. 2A, 2B, 2C. Similarly, the ‘shape-memory’ and topology features of the elastomer-based membrane or of the elastomer-based spring can be achieved using the same manufacturing techniques. Stimulus-responsive shape-memory materials respond to a particular stimulus, such as heat, chemical, magnetic, electric, mechanical and light. The response may be reversible. While in most stimulus-responsive materials, the result is limited to a change in certain physical/chemical properties, stimulus-responsive shape memory materials (SMMs) recover their original shape, after being quasi-plastically distorted. SMMs are ideal for integrated systems, where the materials are actuated and generate a reactive motion. SMMs, include for example shape memory alloys (SMAs) and shape memory polymers (SMPs). SMMs also include ceramics, gels and combinations of these materials. Shape-memory materials and the stimulus to which they respond are included in embodiments of this invention.

The solid particle body can range in size from a few nanometers to a few micrometers and exhibit specific and tunable magnetic properties. The adjustable magnetic features are diamagnetic, paramagnetic, superparamagnetic and ferromagnetic, depending on chemical composition, crystalline structure and size of the particles used. More specifically, representative examples of particle candidates include neodymium (ex., Nd₂Fe₁₄B (“A magnetic membrane actuator in composite technology utilizing diamagnetic levitation,” IEEE Sens. J. 13 (2013), pages 2786-2797), carbon-coated Fe (“Microfabrication of magnetically actuated PDMS-Iron composite membranes,” Microelectr. Engineer. 98 (2012), pages 607-609), iron (II/III) oxides (“Magnetically-actuated artificial cilia for microfluidics propulsion,” Lab Chip. 11 (2011), pages 2002-2010), cobalt alloy(s) (“A facile template-free approach to magnetodriven multifunctional artificial cilia,” Appl. Mater. Interfaces 2 (2010), pages 2226-2230), etc.)

These particles are incorporated into a compatible polymer matrix, such as polydimethylsiloxane (PDMS) (“Magnetically actuated micropumps using an Fe-PDMS composite membrane,” Proc. SPIE Conf. Smart. Struc. Mater. 2006, p. 617213). Additional examples of elastic polymer matrices include but are not limited to poly n-butylacrylate (PnBA) (“Magnetically-actuated artificial cilia for microfluidics propulsion,” Lab Chip. 11 (2011), pages 2002-2010), poly(styrene-block-isoprene-block-styrene) (“A facile template-free approach to magnetodriven multifunctional artificial cilia,” Appl. Mater. Interfaces 2 (2010)), and SU-8 (a commonly used epoxy-based negative photoresist polymer) (“Single cell manipulation using ferromagnetic composite microtransporters,” Appl. Phys. Lett. 96 (2010), 043705).

Specific manufacturing technologies to incorporate particles of interest into magnetoactive elastomers include but are not limited to:
casting as a standalone process or using sacrificial coating (e.g., polyethyleneglycol (PEG); polyvinylacrylate (PVA); or polycarbonate);
molding/casing to produce a pre-determined shape with embedded particles followed by laser-, chemical- or other etching techniques to achieve the desired topology;
photopatterning,
self-assembly under magnetic field, and iv) lithography (“A review of magnetic composite polymers applied to microfluidics devices,” J. Electrochem. Soc. 161 (2014), pages B3173-B3183). The publications cited above are incorporated herein by reference.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A device for implanting in a biological tissue and adapted to move in a viscoelastic media, the device comprising:
a main-body comprising a first material (M1) and having a direction in the viscoelastic media, and wherein the direction of the main body changes upon application of a first stimulus field (SF1) at a first threshold (T1); and
one or more memory shaped elements (MSE) having a first configuration and comprising a second material (M2), said second material comprises an elastomer, and wherein the MSE adopts a second configuration upon application of a second stimulus field (SF2) at a second threshold (T2).

2. The device of claim 1, wherein the second material (M2) is different from the first material (M2≠M1).

3. The device of claim 1, wherein SF1 and SF2 are of the same nature and the same direction; and wherein T2 is larger than T1.

4. The device of claim 1, wherein the material of at least some of the MSEs are different one from another (M2ᵢ≠M2ⱼ, i≠j).

5. The device of claim 1, wherein at least one of M1 and M2 comprises a form of micro- or nano-particles.

6. The device of claim 1, the first or second configuration of the MSE is selected from a group consisting of: an elongated shape, a film, a wire, a string, a strip, a plug, a sheet, a membrane, flagellum, coil, helix, arm, joint and any combination thereof.

7. The device of claim 1, wherein at least one of the MSE is externally attached to the main-body, and adapted to propel the main-body in the viscoelastic media.

8. The device of claim 7, wherein the application of SF2 comprises cycles of the second stimulus field above and below the second threshold (T2).

9. The device of claim 1, wherein the main-body further comprises at least two fins, configured to steer the direction of the main-body.

10. The device of claim 9, wherein the fins comprise the first material (M1).

11. The device of claim 10, wherein the fins comprise a polarity direction at an angle relative to the main-body.

12. The device of claim 9, wherein the fins are externally and symmetrically attached to the main-body.

13. The device of claim 9, wherein the fins are configured to tilt relative to the main-body.

14. The device of claim 1, wherein the main-body further comprises a sealable cavity and when the MSE is in the first configuration the cavity is closed and in the second configuration the cavity is open.

15. The device of claim 14, wherein the sealable cavity is configured to temporarily accommodate at least one of: a therapeutic entity, a therapeutic load, a diagnostic load, or a combination thereof.

16. The device of claim 14, wherein the sealable cavity is configured to temporarily accommodate an explosion material, configured to propel the main-body.

17. The device of claim 14, further comprising a sensitive sealing lid, configured to temporarily seal the cavity; wherein the sensitive sealing lid is configured to be opened responsive to an environmental threshold.

18. The device of claim 14, wherein the MSE is configured as a sealing lid for the cavity; and wherein configuration of the MSE opens and/or closes the sealable cavity.

19. The device of claim 14, wherein the MSE comprises a first arm and pulls and/or pushes a sealing-lid of the cavity upon application of SF2.

20. The device of claim 19, wherein the first arm comprises at least one element selected from: a spring, a helical spring, a leaf spring, a rod, a shaft, a pole and a bar.

21. The device of claim 1, wherein the main-body further comprises a cavity and wherein the MSE comprises a second arm, configured to push a substance accommodated within the cavity out of the cavity upon application of SF2.

22. A system comprising:
The device of claim 1; and
a remote controlling module configured to control the application of SF1 and SF2.

23. The system of claim 22, wherein the remote controlling module comprises at least one inducer for a stimulus field selected from: magnetic, electric, acoustic, ultrasound, heat, X-ray, radio-wave and any combination thereof.

24. The system of claim 22, further comprising a delivery and/or retraction module, configured to deliver and/or retract the device to and/or from a specific location selected from: in vitro, ex vivo, in vivo in a mammalian subject, and in vivo in a human patient.

25. The system of claim 24, wherein the delivery and/or retraction module comprises an attachment element selected from: a magnetizable needle, expandable magnetic element, magnetizable surface, pneumatic element, electromagnetic element, ultrasonic element, deployable mesh, deployable micro-net, suction element, and any combination thereof.

26. The system of claim 22, the remote controlling module comprises a monitoring-device, configured to locate and display location and orientation of the device within the viscoelastic media.

27. A method comprising applying at least one of the stimulus fields (SF) to the device of claim 1 to manipulate motion of the main-body within the viscoelastic fluid of a subject.

28. The method of claim 27, wherein manipulation comprises: steering the main-body to a desired direction via an SF1 corresponding to the lower threshold (T1); and/or propelling the main-body by modifying the configuration of the MSE, via an SF2 corresponding to the second threshold (T2).

29. The method of claim 27, further comprising at least one of:
externally loading the device's cavity with a selected load;
delivering the device into a treated subject;
monitoring the device's location and orientation within the viscoelastic media;
releasing the selected load from the cavity at a desired location;
imaging the subject to locate the device for further diagnostic information; or
retracting the device from a pre-determined location.

30. The method of claim 29, wherein the step of delivering comprises at least one of: injecting, providing for swallow, penetrating via catheter.

31. The method of claim 29, wherein the step of releasing the selected load comprises modifying the configuration of the MSE via the SF2 at the second threshold (T2), such that the cavity's sealing lid is opened.

32. The method of claim 29, wherein the step of releasing the selected load comprises opening the sensitive sealing lid, by providing a selected environmental threshold.
